(19) United States
(12) Patent Application Publication
(10) Pub. No.: US 2020/0113499 A1
(43) Pub. Date: Apr. 16, 2020

(54) MEDICAL EXAMINATION DEVICE

(71) Applicant: MY HOMEDOC LTD., Ra'anana (IL)

(72) Inventors: Gilat SCHREIBER, Beit Hashmonay (IL); Nir RAZ, Hod Hasharon (IL); Shiri RAZ, Hod Hasharon (IL); Mel BERGMAN, Tel-Aviv (IL); Hagai HARDUFF, Binyamina (IL); Oded GOV, Tel-Aviv (IL)

(73) Assignee: MY HOMEDOC LTD., Ra'anana (IL)

(21) Appl. No.: 16/624,966

(22) PCT Filed: Jun. 20, 2018
(86) PCT No.: PCT/IL2018/050680

§ 371 Date: Dec. 20, 2019

(30) Foreign Application Priority Data
Jun. 21, 2017 (IL) 253080
Oct. 18, 2017 (IL) 255121
Nov. 7, 2017 (IL) 255499

(51) Int. Cl. A61B 5/1455; A61B 1/00; A61B 1/04; A61B 1/227; A61B 5/00; A61B 5/01

(52) U.S. Cl. CPC A61B 5/14552; A61B 1/00016; A61B 1/00108; A61B 1/042; A61B 1/227; A61B 5/682; A61B 5/0086; A61B 5/01; A61B 5/6817; A61B 5/6819; A61B 5/0022

(57) ABSTRACT

Provided is directed to a device for medical examination in a subject. The device is configured for easy self-examination of a variety of physiological parameters indicative of disease or physiological irregularities. Examples of such physiological parameters include body temperature indicative of fever, ear pathologies indicative of ear infection, heartbeat, blood oxygen level. While the device of this disclosure may be a stand-alone device, it may be configured, according to embodiments of this disclosure, for association and operation with a mobile communication device, in particular a smartphone.

### TECHNOLOGICAL FIELD

This disclosure concerns a device for use in medical examination, particularly a device for home medical examination.

### BACKGROUND ART

References considered to be relevant as background to the presently disclosed subject matter are listed below:
WO2013/156999
WO2009/094132
WO2004/053638

Acknowledgement of the above references herein is not to be inferred as meaning that these are in any way relevant to the patentability of the presently disclosed subject matter.

### BACKGROUND

While in the past diagnosis of medical conditions and medical examination was done exclusively in a clinic or medical laboratory, in recent years there is a growing reliance in medical practice on examination carried out at home. In order to perform such examination, a subject needs to be provided with a suitable and easy to use device. A medical examination system for home use that is coupled to a mobile device has been disclosed in WO2013/156999 and WO2009/094132.

### GENERAL DESCRIPTION

The present disclosure is directed to a device for medical examination in a subject. The device is configured for easy self-examination of a variety of physiological parameters indicative of disease or physiological irregularities. Examples of such physiological parameters include body temperature indicative of fever, ear pathologies indicative of ear infection, heartbeat, blood oxygen level. While the device of this disclosure may be a stand-alone device, it may be configured, according to embodiments of this disclosure, for association and operation with a mobile communication device, in particular a smartphone. The mobile communication device may then serve as the medical device's interface as well as for processing of the physiological parameters, converting into data and transmitting such data to a caregiver, medical center or data repository.

One aspect of this disclosure concerns a device that comprises a body having integral sensors and optical assembly for acquiring medical data. The body houses one or more communication modules that can transmit the acquired data, e.g. by means of wireless communication, to another device, a data repository, etc.

According to one embodiment, the device is provided with an attachment arrangement configured for physical attachment to a mobile communication device. The sensors comprise temperature sensor (e.g. an infrared-based sensor) and an acoustic sensor, the former serving for recording temperature of the body surface, and the latter serving for auscultation, particularly with regards to acoustic manifestations of the heart, lungs, and digestive system. The acoustic sensor may be a stethoscope that can record heart-related data when positioned against a body surface.

By one embodiment, the attachment arrangement comprises at least two gripping elements configured to tightly hold or engage with portions (typically, but not exclusively opposite portions) of the mobile device's frame and thereby tightly attach to the mobile communication device in a detachable manner.

By one embodiment, at least one of the gripping elements is formed at the end of an arm that extends from the body and can slidably extend to an extended and retracted position and is biased into the latter. At least one other gripping element is typically integral with the body (and may be formed as an appropriately configured abutment) and the bias of the arm against the body then provides for tight attachment. In other embodiments, both gripping elements may be formed at an end of such biased arm. The temperature sensor, by one embodiment, is housed within an abutment, abutting off a surface of said body. By one exemplary embodiment, this abutment serves also as integral and fixed gripping element (functioning together with the gripping element on said arm for tight attachment to a mobile communication device).

The body of the device, by one embodiment, comprises a generally planar forehead-engagement surface for physical engagement with the subject's forehead. This portion defines a first plane and said temperature sensor is an infrared sensor typically positioned at a distance from said first plane, with the center of its field of view defining a line normal to said first plane. The distance is chosen to ensure that the surface area ‘viewed’ by the sensor on the forehead is sufficient for reliable, repeatable and independent on exact positioning.

The optical assembly in the device is configured to allow body surface or cavity measurement (e.g. of the ear, throat, or nose), by permitting acquisition of an optical image of the tissue surface to be examined. The optical assembly is also configured for coupling to a disposable element that envelopes the exterior housing of the optical assembly and permits repeated clean or sterile examination of the ear canal (otoscopic measurement), throat, etc. This element may be a disposable hollow medical element configured for safe insertion into the ear canal or configured as a disposable tongue depressor. The optical assembly typically defines an optical axis between an image sensor and the target tissue (the direction from the sensor to the target tissue will be referred to herein as “forward direction” and the opposite direction as “rearward direction”, the rearward direction being that in which the image propagates from the tissue to the sensor).

The optical assembly may also comprise illumination means for transmitting illuminating light in the forward direction to the tissue to be examined. The illuminating light, on the one hand, and the acquired image on the other hand may propagate in opposite directions along the same optical axis—in respective forward and rearward directions. The optical assembly may, thus, include also a beam splitter that permits to reflect light from an illumination source positioned off the optical axis (typically emitting light in a direction normal to the optical axis) in the forward direction, on the one hand, and to permit the acquired image to propagate to the image senor.

The optical assembly also includes one or more lenses disposed along the optical axis.

In the typical embodiment where the medical device works in association with a mobile communication device, the optical sensor/camera used for image acquisition is typically that of the mobile communication device. Cameras of different mobile communication devices vary from one another in their fundamental optical parameters including different lenses and sensors. Accordingly, the optical assembly is designed to permit for tolerance between different mobile communication devices' cameras, permitting proper focus and picture quality on the sensor regardless of the fine optical parameters and the depth of the sensor.

The optical assembly may be formed as part of the body or alternatively may be formed in a movable portion, e.g. slidable, with respect to the body. This is intended for fine-tuning of the optical assembly's position for proper alignment with the camera of the mobile communication device. This portion typically protrudes laterally from the lateral edge of the base body.

The device, by one embodiment, comprises a pulse oximeter, typically including a fingertip-accommodating depression formed on a surface of the body and configured to accommodate the tip of the subject's finger, with the pulse oximeter being positioned at the bottom of said depression.

Another aspect of this disclosure is a device for medical examination in a subject which includes a body with integral sensors for acquiring medical-related physiological data and housing one or more communication modules for transmission of acquired data, for example, through wireless communication, to another device or data repository. The sensors comprise, as noted above, infrared sensor and acoustic sensor, e.g. a stethoscope. The body generally comprises planar forehead-engagement of the subject's forehead that defines a first plane. The infrared sensor is positioned in the body so that the distance from said first plane and the center of its field of view defines a line that is normal to said first plane. In this manner, once the surface is positioned against the forehead, the sensor can detect infrared radiation while on a portion of the forehead, which may be converted into temperature-related data.

By a typical embodiment, the acoustic sensor (e.g. a stethoscope) is embedded in the forehead-engagement surface for acquiring auscultation data, once the said surface is placed against an intended tissue surface, e.g. the chest for acquiring pulse related data.

A further aspect of this disclosure concerns a device for medical examination in a subject that comprises an optical assembly configured for association with acquiring medical-related physiological data; an attachment arrangement for fixing said device to a mobile communication device; and an arrangement configured for associating the optical assembly with the camera of the mobile communication device. The optical assembly may be configured to allow body surface or cavity measurement (e.g. of the ear, throat, or nose).

The optical assembly may be configured for coupling to a disposable hollow medical element, for example an ear examination medical elements and may also comprise a light source configured to illuminate a target tissue. The attachment arrangement, may according to several embodiments of this aspect, be any one of those noted above. Furthermore, this device, may comprise a body with integral sensors for capturing medical-related physiological data and housing one or more communication modules (typically, but not exclusively, one) for transmitting acquired data (e.g. through wireless communication). The sensors may be any one of those noted above.

The attachment arrangement for fixing said device to a mobile communication device may comprises at least two gripping elements configured for tight holding of portions of the mobile device's frame, of the kind described above. The arrangement configured for associating the optical assembly with the camera of the mobile communication device may, by some embodiments, comprise a mechanism for displacing the optical assembly in a plane parallel to that defined by the mobile communication device to bring it into alignment with said camera. In addition to permitting displacement and alignment in 2-dimensions, said arrangement may, by certain embodiments, also have the ability for displacement in 3-dimensions, namely also comprising a mechanism for displacement along an axis normal to the plane of the mobile communication device. The optical assembly may, by certain embodiments, be mounted on a portion movable with respect to a main body of the device, permitting 2-D and, at times, 3-D displacement.

In some embodiments the movable portion has a lens depression configured for accommodating a protruding camera lens of the mobile device.

The analysis of the physiological data picked by the sensors may be performed locally in the mobile communication device. For this purpose the mobile communication device may comprise a dedicated software or a hardware utility for receiving the acquired data and analyzing the data. The analysis may also be carried out remotely in a remote server or by means of cloud computing. The acquired raw data or the initially processed data (after initial processing, e.g. compressing, in the mobile communication device) may then be transmitted to the “cloud” for analysis. The results of the analysis may be transmitted back to the mobile communication device or to a medical center or care-giver.

The medical device of this disclosure may also be capable of communication with other devices, such as, for example, a voice input/output device that may guide that medical examination through computerized voice commands. The medical device of this disclosure may, also, be capable of being activated by a communication device including, but not limited to, a mobile communication device.

### EMBODIMENTS

Defined below are some non-limiting exemplary embodiments a device of the present disclosure, defined in the form of numbered clauses:

1. A device for medical examination in a subject comprising

a body with integral sensors and is associated with or comprises an optical assembly for capturing medical-related physiological data and housing one or more communication modules for transmitting acquired data (e.g. through wireless communication); and

an attachment arrangement configured for physical attachment to a mobile communication device;

wherein the sensors comprise a temperature sensor, e.g. an infrared sensor, and an acoustic sensor configured for recording physiological data.

2. The device of clause 1, wherein the attachment arrangement comprises at least two gripping elements configured for tight holding of opposite portions of the mobile device's frame.

3. The device of clause 2, wherein at least one of the gripping elements being at an end of an arm extending from the body and slidable between extended and retracted positions and biased into the latter.

4. The device of clause 3, wherein one of the gripping elements is configured by an abutment on a surface of said body and the temperature sensor is housed within said abutment.

5. The device of any one of clauses 1-4, wherein the optical assembly is configured for coupling to a disposable hollow medical element for ear examination.

6. The device of any one of clauses 1-5, wherein the optical assembly is mounted on a portion moveable with respect to the body.

7. The device of clause 6, wherein the movement of the movable portion is configured so as to fit the optical assembly over a camera of the mobile device.

8. The device of clauses 6 or 7, wherein the movable portion is protruding from a lateral edge of the body.

9. The device of any one of clauses 6-8, wherein the movable portion has a lens depression configured for accommodating a protruding camera lens of the mobile device.

10. The device of any one of clauses 1-9, wherein the optical assembly comprises a light source configured to illuminate a target tissue.

11. The device of any one of clauses 1-10, wherein the body comprises a generally planar forehead-engagement surface for physical engagement with the subject's forehead, said portion defining a first plane; and wherein the temperature sensor is an infrared sensor positioned at a distance from said first plane and defining a line at the center of its field of view that is normal to said first plane.

12. The device of any one of clauses 1-11, comprising pulse oximeter.

13. The device of clause 12, comprising a fingertip-accommodating depression, configured to accommodate a tip of the subject's finger; the pulse oximeter being positioned at a bottom of said depression.

14. A device for medical examination in a subject comprising

a body with integral sensors for acquiring medical-related physiological data and housing one or more communication modules for transmitting acquired data (e.g. through wireless communication);

wherein the sensors comprise an infra-red sensor and an acoustic sensor configured for recording a physiological data;

a generally planar forehead-engagement surface for physical engagement with the subject's forehead, said portion defining a first plane; and

wherein the infrared sensor is positioned at a distance from said first plane and defining a line at the center of its field of view that is normal to said first plane.

15. The device of clause 14, wherein the acoustic sensor is part of the forehead engagement surface.

16. The device of clauses 14 or 15, wherein the acoustic sensor is a stethoscope.

17. The device of any one of clauses 14-16, wherein the device comprise an attachment arrangement configured for physical attachment to a mobile communication device (e.g. a smartphone).

18. The device of clause 17, wherein the attachment arrangement comprises at least two gripping elements configured for tight holding of opposite portions of the mobile device's frame.

19. The device of clause 18, wherein at least one of the gripping elements being at an end of an arm extending from the body and slidable between extended and retracted positions and biased into the latter.

20. The device of clauses 18 or 19, wherein one of the gripping elements is configured by an abutment on a surface of said body and the infra-red sensor is housed within said abutment.

21. The device of any one of clauses 14-20, wherein the body is associated with integral optical assembly.

22. The device of clause 21, wherein the optical assembly is configured for coupling to a disposable hollow medical element for ear examination.

23. The device of clauses 21 or 22, wherein the optical assembly is mounted on a portion moveable with respect to the body.

24. The device of clause 23, wherein the movement of the movable portion is configured so as to fit the optical assembly over a camera of the mobile device.

25. The device of clauses 23 or 24, wherein the movable portion is protruding from a lateral edge of the base body.

26. The device of any one of clauses 23-25, wherein the movable portion has a lens depression configured for accommodating a protruding camera lens of the mobile device.

27. The device of any one of clauses 14-26, comprising pulse oximeter.

28. The device of clause 27, comprising a fingertip-accommodating depression, configured to accommodate a tip of the subject's finger; the pulse oximeter being positioned at a bottom of said depression.

29. A device for medical examination in a subject comprising

an optical assembly configured for acquiring medical-related physiological data;

an attachment arrangement for fixing said device to a mobile communication device; and

an arrangement configured for associating the optical assembly with the camera of the mobile communication device.

30. The device of clause 29, wherein the attachment arrangement comprises at least two gripping elements configured for tight holding of opposite portions of a frame of the mobile communication device.

31. The device of clause 30, wherein at least one of the gripping elements being at an end of an arm extending from the body and slidable between extended and retracted positions and biased into the latter.

32. The device of clause 31, wherein one of the gripping elements is configured by an abutment on a surface of said body and the temperature sensor is housed within said abutment.

33 The device of any one of clauses 29-32, wherein the arrangement comprises a mechanism for displacing the optical assembly in a plane parallel to that defined by the mobile communication device to bring it into alignment with said camera.

34. The device of any one of clauses 29-33, wherein the optical assembly has a lens depression configured for accommodating a protruding camera lens of the mobile device.

35. The device of any one of clauses 29-34, wherein the device comprise a body and the optical assembly is mounted on a portion movable with respect to the body.

36. The device of any one of clauses 29-35, comprising a body with integral sensors for capturing medical-related physiological data and housing one or more communication modules for transmitting acquired data (e.g. through wireless communication).

37. The device of any one of clauses 29-36, wherein the optical assembly is configured for coupling to a disposable hollow medical element.

38. The device of claim 37, wherein the disposable hollow medical element is suitable for ear examination.

39. The device of any one of clauses 29-38, wherein the optical assembly comprises a light source configured to illuminate a target tissue.

39. The device of any one of claims 29-39, having one or more of the limitations specified in clauses 1-28.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B are isometric views of a device according to an embodiment of this disclosure showing the device with the extendable arm in an extended position and retracted position respectively.

FIG. 2A-2B shows the device of FIG. 1 with a disposable element for ear examination, uncoupled and coupled to the optical assembly respectively.

FIGS. 3A-3C are perspective views and FIG. 3D is a side view of the device of FIGS. 1 and 2 coupled to a mobile communication device, e.g. a smartphone.

FIGS. 4A-4B is a schematic illustration of the vertical displacement of the optical assembly with a disposable ear examination element attached thereto.

FIG. 5 is a schematic illustration of the optical elements of the optical assembly.

FIG. 6 is a schematic illustration of the optical path of light between an image sensor, e.g. a camera, and the target tissue.

FIG. 7 is a block diagram representing a data flow between the device of the present disclosure and the mobile communication device.

FIG. 8 is a schematic block diagram of a system that includes the device of this disclosure.

### DETAILED DESCRIPTION OF EMBODIMENTS

In the following description, this disclosure will be illustrated by specific embodiments of a device for attachment to a smartphone, utilizing the smartphone's camera for image acquisition. As will be understood, this is but an example of the broader context of this disclosure.

In the text herein, terms like vertical, lateral, upward, downward, rear surface, front surface, are being used to match the views seen in the attached Figures. It is to be understood that these terms are used for the purpose of convenience only and do not necessarily have functional significance.

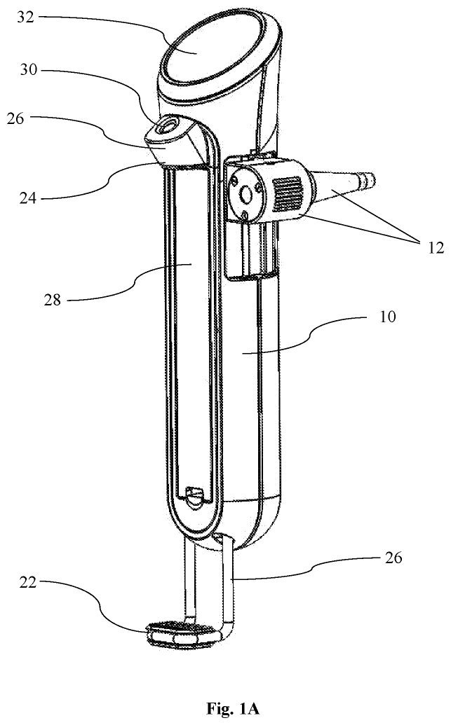

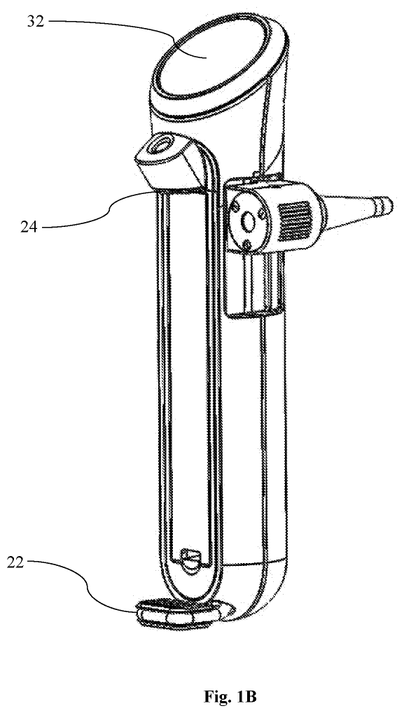

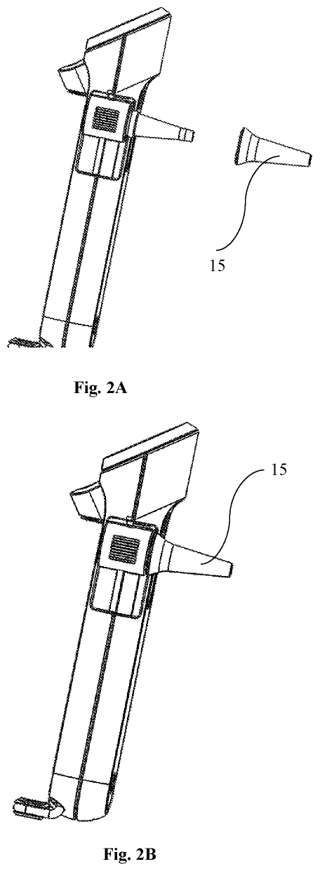

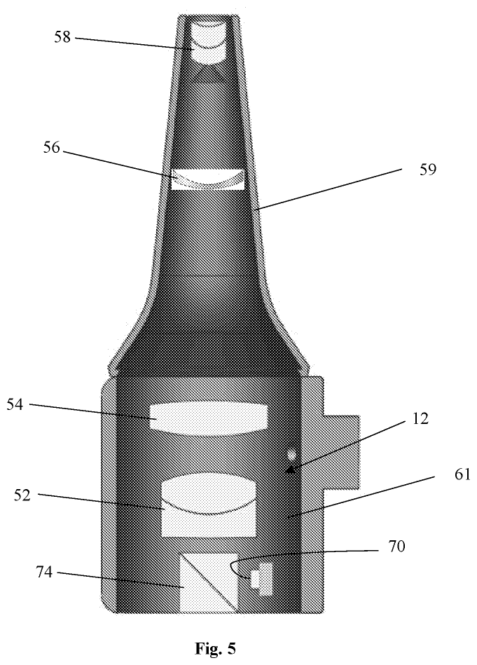

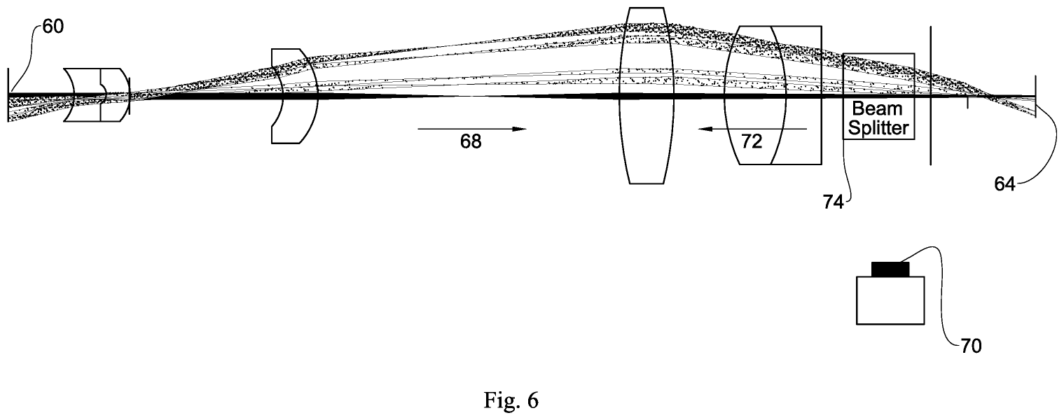

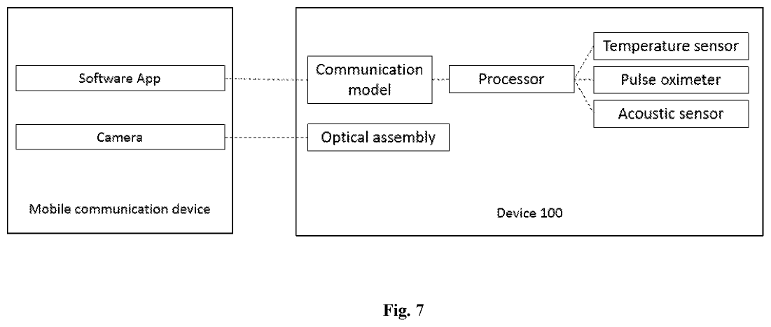

Reference is first being made to FIGS. 1A-1B showing a generally elongated body 10 having some integral sensors (to be described below) associated with optical assembly 12. The optical assembly is elongated and defines an optical axis with several lenses disposed along the axis as illustrated schematically in FIGS. 5 and 6 and described further below. The sensors and the optical assembly are configured for acquiring medical-related physiological data. A disposable element 15 is configured to be coupled with the optical assembly as can best be seen in FIGS. 2A-2B. The disposable element is typically clean and is typically intended for a single use. The body houses at least one communication module (not shown) coupled to the sensor and configured for transmitting acquired data through wireless (e.g. Bluetooth) communication to the mobile communication device.

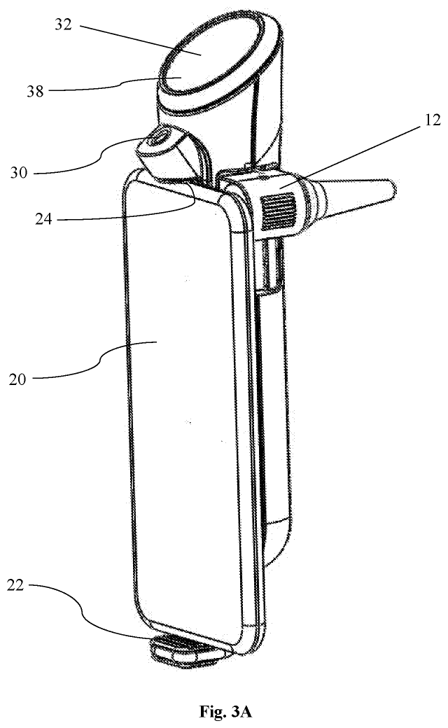

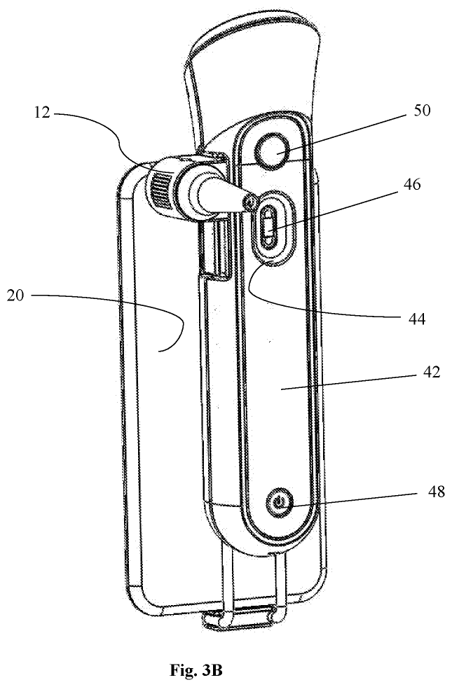

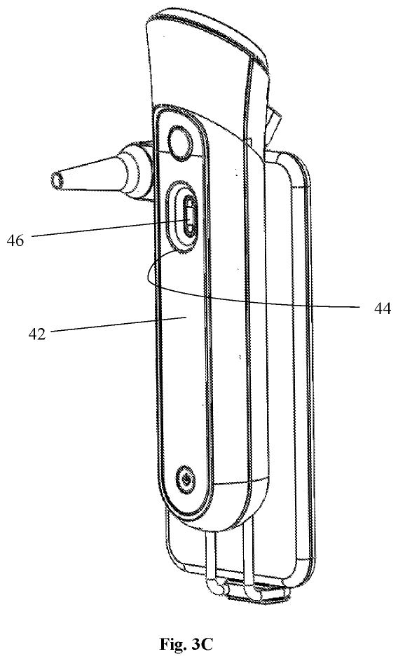

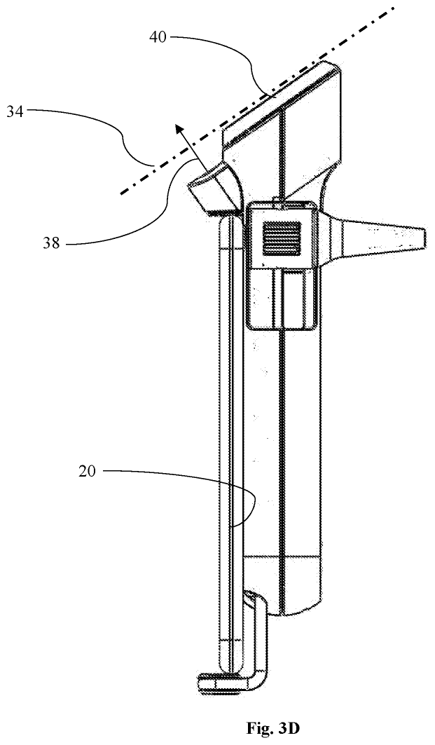

The device includes an attachment arrangement configured for physical attachment to a mobile communication device 20, e.g. a smartphone, in the manner seen in FIGS. 3A-3B. The attachment arrangement includes two gripping elements 22, 24 configured for tight holding of opposite portions of the mobile device frame, in a manner seen in FIGS. 3A-3D. At least one of these gripping elements is at the end of an arm 26 which extends from the body and is slidable between extended and retracted positions, biased into the latter and, accordingly, ensures tight attachment to the communication device frame. The other gripping elements 24 is part of abutment 26 protruding out of rear surface 28 of the body. This abutment (best seen in FIG. 3A), also houses a temperature sensor, which in this example is an infrared sensor 30 (as will be described later).

Formed at an upper end of the device is a forehead-engagement surface 32 for physical engagement with the subject's forehead and defines a first plane, represented in FIG. 3D by a dashed line 34. The infrared sensor 30 has a field of view with its center, represented by arrow 38 in a direction normal to said first plane. In this way, once the forehead-engagement surface is positioned against the forehead, the sensor can sense infrared radiation from the forehead that can then be processed into a measure of temperature.

The forehead-engagement surface, in a device of this embodiment, includes also an acoustic sensor 40 (in the form of a stethoscope) with a flexible membrane and a mechanical sensor associated therewith (not shown) for recording auscultation data upon engagement of the surface with the forehead. Thus, once so engaged, pulse and temperature data may be recorded simultaneously.

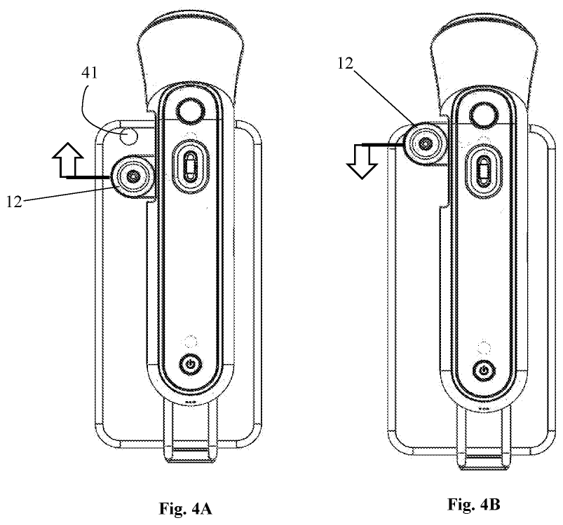

The device also includes an optical assembly 12 housed within a laterally protruding portion, configured to permit vertical displacement, in a manner represented in FIGS. 4A-4B. This vertical sliding displacement enables to fine-tune the position of the optical assembly over an image sensor, e.g. camera 41 of the mobile communication device, to align its optical assembly with the camera. It should be noted that the camera's position in mobile devices vary from one another and in this manner the medical device may be used with a wide variety of mobile communication devices.

Front surface 42 of the body includes depression 44 configured to accommodate the tip of a subject's finger, and positioned within the center of the depression is a pulse oximeter 46 that, once a finger is positioned within the depression, can measure oxygen saturation level. The device also has an on/off switch 48 at the bottom of its front surface. In use, measurement of the different physiological data may be done in a sequence, typically guided by an application running on the mobile communication device which is triggered into operation upon powering the device through the on/off switch 48. The software application on the device may also guide the user through the examination sequence and may also be configured through wireless communication to activate the various sensors and ready them for the respective data acquisition. Alternatively, the software application may guide the user through the examination sequence, but the initiation of the different examinations will be through operation of button 50.

Reference is now being made to FIGS. 5 and 6 showing a schematic illustration of the optical assembly 12 including, in this example, four lens groups 52, 54, 56, 58, two of which (52 and 58) consisting of two lenses, each and the other two of one lens each. The optical assembly is formed by two integral parts, including a first, cylindrical bases 59 and a second, generally frusto-conical hollow part 61. All these lenses are part of the optical arrangement and define an optical path in which the acquired tissue image 60 propagating in the rearward direction of arrow 62 to the image sensor 64 within the device's camera.

For illumination of the target tissue, e.g. surface of the ear canal, a light source 70 is provided which transmits light in a direction normal to the optical path which is reflected into the forward direction of the optical path, as represented by arrow 72, by means of a beam splitter 74.

Reference is now made to FIG. 7 that shows a schematic block diagram representing the data flow between the device and the mobile communication device. The sensors of the device are acquiring signals that are processed in a processor within the device. The processed data is then transmitted by communication module to the mobile communication device and processed there by a software application. The optical arrangement defines an optical path in which the acquired tissue image propagates to the mobile communication device's camera.

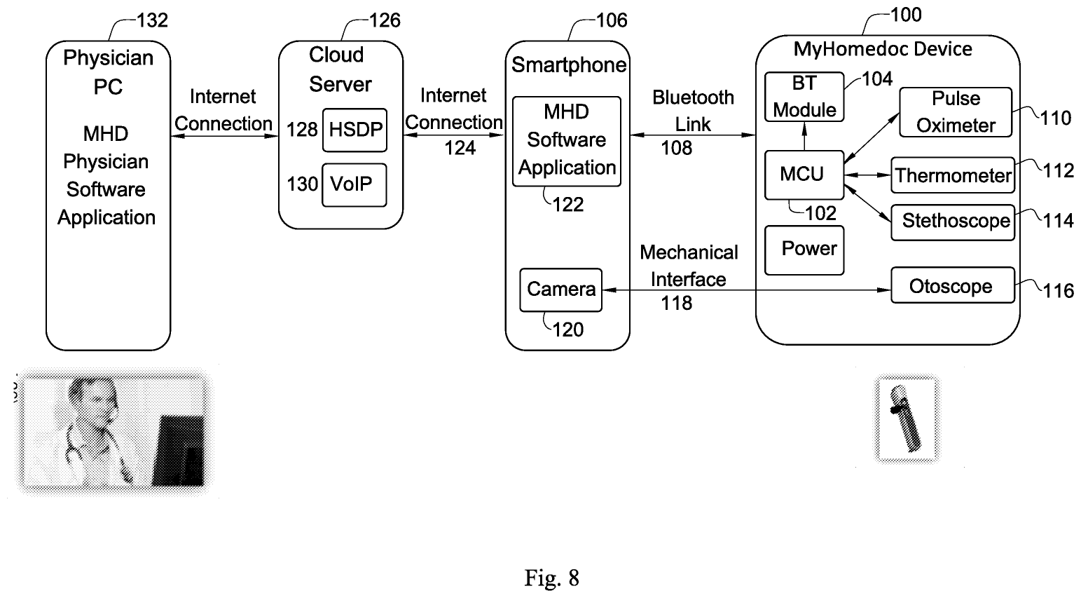

FIG. 8 is a schematic block-diagram of a system that includes a device of this disclosure. The device, identified as block 100 includes a processor 102, a Bluetooth transceiver 104, for communication with a mobile communication device 106 (e.g. a smartphone), via a Bluetooth-established link 108. The device has three sensing modules, including a pulse oximeter 110, thermometer 112 and stethoscope 114 as well as an otoscope unit 116 that has a mechanical interface 118 with the camera 120 of the mobile communication device 106. The medical device may be similar to that described above in FIGS. 1-6.

Operating on the mobile communication device is a medical health diagnostic (MIND) software utility 122 that is linked, via an internet connection 124 to a cloud server 126 that runs a health analysis software 128 that may include, that may be voice operated through a voice-over-IP (VoIP) interface module 130 that may receive and/or issue voice input/output. The cloud server 126 may be configured to transmit the analyzed health data to a medical center or a health-giver computer 132 and may also serve as a channel for medical instruction or input from the medical center or a health-giver back to the mobile communication device 106.

1. A device for medical examination in a subject comprising
a body with integral sensors and is associated with or comprises an optical assembly for acquiring medical-related physiological data and housing one or more communication modules for transmitting acquired data; and
an attachment arrangement configured for physical attachment to a mobile communication device;
wherein the sensors comprise a temperature sensor and an acoustic sensor configured for recording physiological data.

2. The device of claim 1, wherein the attachment arrangement comprises at least two gripping elements configured for tight holding of opposite portions of the mobile device's frame.

3. The device of claim 2, wherein at least one of the gripping elements being at an end of an arm extending from the body and slidable between extended and retracted positions and biased into the latter.

4. The device of claim 3, wherein one of the gripping elements is configured by an abutment on a surface of said body and the temperature sensor is housed within said abutment.

5. The device of claim 1, wherein the optical assembly is configured for coupling to a disposable hollow medical element for ear examination.

6. The device of claim 1, wherein the optical assembly is mounted on a portion moveable with respect to the body;
wherein the movement of the movable portion is configured so as to fit the optical assembly over a camera of the mobile device.

7. (canceled)

8. The device of claim 6, wherein the movable portion is protruding from a lateral edge of the body.

9. The device of claim 6, wherein the movable portion has a lens depression configured for accommodating a protruding camera lens of the mobile device.

10. The device of claim 1, wherein the optical assembly comprises a light source configured to illuminate a target tissue.

11. The device of claim 1, wherein the body comprises a generally planar forehead-engagement surface for physical engagement with the subject's forehead, said portion defining a first plane; and
wherein the temperature sensor is an infrared sensor positioned at a distance from said first plane and defining a line at the center of its field of view that is normal to said first plane.

12-13. (canceled)

14. A device for medical examination in a subject comprising
a body with integral sensors for acquiring medical-related physiological data and housing one or more communication modules for transmitting acquired data;
wherein the sensors comprise a temperature sensor and an acoustic sensor configured for recording a physiological data;
a generally planar forehead-engagement surface for physical engagement with the subject's forehead, said portion defining a first plane; and
wherein the infrared sensor is positioned at a distance from said first plane and defining a line at the center of its field of view that is normal to said first plane.

15. The device of claim 14, wherein the acoustic sensor is part of the forehead engagement surface.

16. (canceled)

17. The device of claim 14, wherein the device comprises an attachment arrangement configured for physical attachment to a mobile communication device;
wherein the attachment arrangement comprises at least two gripping elements configured for tight holding of opposite portions of the mobile device's frame;
wherein at least one of the gripping elements being at an end of an arm extending from the body and slidable between extended and retracted positions and biased into the latter; and
wherein one of the gripping elements is configured by an abutment on a surface of said body and the infra-red sensor is housed within said abutment.

18-22. (canceled)

23. A device for medical examination in a subject comprising
an optical assembly configured for acquiring medical-related physiological data;
an attachment arrangement for fixing said device to a mobile communication device; and
an arrangement configured for associating the optical assembly with the camera of the mobile communication device.

24. The device of claim 23, wherein the attachment arrangement comprises at least two gripping elements configured for tight holding of opposite portions of a frame of the mobile communication device;
wherein at least one of the gripping elements being at an end of an arm extending from the body and slidable between extended and retracted positions and biased into the latter; and
wherein one of the gripping elements is configured by an abutment on a surface of said body and the temperature sensor is housed within said abutment.

25-26. (canceled)

27. The device of claim 23, wherein the arrangement comprises a mechanism for displacing the optical assembly in a plane parallel to that defined by the mobile communication device to bring it into alignment with said camera.

28. The device of claim 23, wherein the optical assembly has a lens depression configured for accommodating a protruding camera lens of the mobile device.

29. The device of claim 23, wherein the device comprises a body and the optical assembly is mounted on a portion movable with respect to the body.

30. The device of claim 23, comprising a body with integral sensors for capturing medical-related physiological data and housing one or more communication modules for transmitting acquired data.

31. The device of claim 23, wherein the optical assembly is configured for coupling to a disposable hollow medical element;
wherein the disposable hollow medical element is suitable for ear examination; and
wherein the optical assembly comprises a light source configured to illuminate a target tissue.

32-33. (canceled)
